(19) United States
(12) Patent Application Publication
(10) Pub. No.: US 2026/0224443 A1
(43) Pub. Date: Aug. 6, 2026

(54) MULTI-CHAMBER BAG FOR PRODUCING DIALYSATE

(71) Applicant: Fresenius Medical Care Deutschland GmbH, Bad Homburg (DE)

(72) Inventors: Paul Gastauer, Bad Homburg (DE); Franz Kugelmann, Bad Homburg (DE); Christian Schley, Bad Homburg (DE); Pascal Joachimsky, Bad Homburg (DE)

(21) Appl. No.: 19/153,228

(22) PCT Filed: Jan. 31, 2024
(86) PCT No.: PCT/EP2024/052298

§ 371 Date: Aug. 1, 2025

(30) Foreign Application Priority Data
Feb. 2, 2023 (DE) 102023102598.8

(51) Int. Cl. A61J 1/20; A61J 1/10; A61J 1/14; A61M 1/16; B65B 3/00; B65B 3/02; B65B 61/16

(52) U.S. Cl. CPC A61J 1/2093; A61J 1/10; A61J 1/1462; A61J 1/2024; A61M 1/1668; B65B 3/003; B65B 3/02; B65B 61/16; A61M 2207/00

(57) ABSTRACT

The present disclosure relates to a multi-chamber bag for producing a medical solution including at least two chambers that are connected to each other via a peel seam and that are delimited by a respective sealing seam at a first side and a second side opposite the first side, wherein the multi-chamber bag further includes a third side formed by a folded-over film, on which at least one connection is formed on the third side by means of butt welding.

### CROSS-REFERENCE TO RELATED APPLICATIONS

The present application is the national stage entry of International Patent Application No. PCT/EP2024/052298, filed Jan. 31, 2024, and claims priority to Application No. DE 102023102598.8, filed in the Federal Republic of Germany on Feb. 2, 2023, the contents of which are hereby incorporated by reference.

### TECHNICAL FIELD

The present disclosure relates to a multi-chamber bag for producing dialysate and to a method for producing such a multi-chamber bag.

### BACKGROUND

Bags containing prefabricated dialysate or at least partial components thereof are frequently used for dialysis. For example, for reasons of storage stability, it may be advantageous to store partial components of a solution, such as dialysate, separately and only mix them immediately before using the solution.

Multi-chamber bags are often used for this purpose, which contain separate sub-components in separate chambers, which are mixed together immediately before a solution is used.

For example, such multi-chamber bags could have two different solutions, a solution and a dry concentrate, a solution and a pasty concentrate or any other contents. Examples include solutions for peritoneal dialysis or hemofiltration as well as preparations for parenteral nutrition.

EP 0 935 967 describes a solution for peritoneal dialysis consisting of two individual solutions provided in a two-chamber bag. The first individual solution comprises calcium ions, other electrolyte salts and glucose in an osmotically effective concentration. The second individual solution comprises bicarbonate and the salt of a weak acid with pka<5. Before use, both chambers must be mixed to obtain the ready-to-use peritoneal dialysis solution.

WO 2003/059417 describes a two-part dialysis solution comprising a first component comprising a bicarbonate concentrate, a second component comprising an electrolyte concentrate, which is provided in a two-compartment bag and is mixed immediately before use.

Conventional multi-chamber bags each have a connection or filling port for filling the respective chambers by means of which each chamber can be filled. The connectors, filling spouts and sealing caps required for this complicate the production and application of such multi-chamber bags.

### SUMMARY

In some implementations, devices, system, and methods of the present disclosure can mitigate or even completely eliminate the problems of the prior art. For example, an advantage of the present disclosure is to provide a simplified and more cost-effective multi-chamber bag and an associated manufacturing process.

Accordingly, a multi-chamber bag for producing a medical solution (e.g., dialysate) is provided with at least two chambers, which are connected to each other by a peel seam and which are each limited by a seal seam on a first and a second opposite side of the bag. The multi-chamber bag also comprises a third side formed by a folded-over film, on which at least one connection is arranged by means of butt welding.

Attaching the connection to the third side can provide the advantage that the connection sits on an outermost layer of the folded-over film and is not integrated into the folded-over film or its layer structure or is interposed between two films.

In some examples, the term butt welding refers to a method in which two end faces are butt welded together in contact with each other. Butt welding is also known as mirror welding.

For example, the connection has a tube section. Firstly, the tube section is widened, (e.g., in a tulip shape) at one end of the tube section in order to create a larger end face in the manner of a flange. The end face of the tube section is then placed on a surface, such as a film, and welded thereto.

In this procedure, the end face of the tube section then sits on the surface of the film and is not integrated into it, for example in the case of a multi-layer film it is arranged between the layers of the multi-layer film or interposed between two films.

Butt welding can be used to create strong and resilient joints.

A plurality of connections can be arranged on the third side, of which at least one, (e.g., all) are arranged on the film by means of butt welding.

According to one embodiment, the multi-chamber bag comprises a sealing seam on a fourth side opposite the third side, e.g., with a suspension eyelet or a plurality of suspension eyelets. The width of the sealing seam can be such that the suspension eye(s) is/are completely surrounded by the sealing seam.

In some embodiments, the multi-chamber bag, or at least one chamber of the multi-chamber bag, does not have a filling port for filling at least one chamber of the multi-chamber bag, e.g., in the form of a connection, port, or connector.

Such a filling port can be understood as an access to a receiving area of the bag, which is designed to fill the receiving area of the bag before the bag or bag system is delivered to an end user or before the bag is used in peritoneal dialysis. After filling, the filling port is usually closed permanently, for example with a sealing cap.

Such a filling port is not required, since a multi-chamber bag as described herein can be filled using a method as described herein before the bag is closed.

It is therefore not necessary to feed a liquid into a chamber of the bag via a port, for example, but the chamber is only closed and formed after it has been filled.

It is conceivable that the multi-chamber bag thus has only one connection for removing contents from the bag, wherein, e.g., at least one chamber of the multi-chamber bag does not have its own connection, port, or connector. The content of this chamber can, e.g., only reach the outside via the connection of another chamber of the multi-chamber bag, for example after a peel seam separating the chambers has been opened.

According to one embodiment, the multi-chamber bag comprises a first chamber and a second chamber, wherein the first chamber is limited by the third side having the connection and the second chamber can be connected to the first chamber by opening the peel seam, so that the contents of the second chamber can only be transferred to the outside via the first chamber and the connection on the third side of the bag.

The second chamber can be limited exclusively by the peel seam, the sealing seams on the first and second sides and the sealing seam on the fourth side.

In some embodiments, a longitudinal axis of the peel seam extends at right angles or substantially at right angles to a longitudinal axis of the multi-chamber bag. This makes it easy to form two chambers of different sizes.

Alternatively, the peel seam can also extend parallel to a longitudinal axis of the multi-chamber bag.

According to one embodiment, the at least two chambers of the bag each have a content, which is designed to yield ready-to-use dialysate when the content is combined with a content of the other chamber of the multi-chamber bag.

For example, at least one chamber can contain an acidic concentrate and the other chamber can contain an ionic solution. At least one chamber may contain bicarbonate and/or an osmotic agent, for example a saccharide.

The present disclosure further relates to a method for producing a multi-chamber bag, e.g., a multi-chamber bag as described herein, the method including:
a) providing a flat film with two outer edges, wherein the flat film can be a multi-layer film or a single-layer film;
b) fixing at least one connection to the flat film by means of butt welding, and, e.g., producing an opening of the flat film in the region of the connection, for example by punching out or using a hot mandrel;
c) producing a film tube out of the flat film, e.g., by means of a forming shoulder over which the flat film extends and by sealing the outer edges of the flat film together;
d) producing a peel seam between two layers of the flat film of the film tube, which detachably joins the two layers of the flat film to one another, which peel seam extends between two outer edges of the film tube along a longitudinal direction of the film tube, whereby at least two receiving areas are created that are separated from each other by means of the peel seam;
e) producing a sealing seam that extends perpendicular or substantially perpendicular to the peel seam, whereby a receiving pocket is produced in each of the two receiving areas;
f) filling the two receiving pocket along the longitudinal direction of the film tube, e.g., with two different contents; and
g) closing the two receiving pockets by producing an additional sealing seam that extends perpendicular or substantially perpendicular to the peel seam, whereby the receiving pockets are closed and at least two completely closed chambers are formed.

The following parameters can be used for butt welding in step b) above:

| | |
|---|---|
| Welding temperature: | 150° C. ≤ T ≤ 300° C. |
| Welding time: | 0.7 s ≤ t ≤ 2 s |
| Welding pressure: | 0.1N/mm² ≤ σ ≤ 2N/mm² |

The following parameters are used in some embodiments:

| | |
|---|---|
| Welding temperature: | 190° C. ≤ T ≤ 250° C. |
| Welding time: | 0.9 s ≤ t ≤ 1.5 s |

Welding pressure: 3000 N where A=6300 mm²→0.48 N/mm²

In some embodiments, multi-chamber bags are produced continuously along a continuous film tube, in each case connected to each other via a sealing seam, which multi-chamber bags are separated from each other by separation along a sealing seam extending perpendicular or substantially perpendicular to the peel seam.

In other words, a continuous film tube can be produced, which is filled in the longitudinal direction of the tube and which is portioned into different bags by sealing in the transverse direction.

This means that when the tube is filled, e.g., two parallel pillars of filling material are created, separated by the peel seam, which are divided into a plurality of multi-chamber bags by transverse sealing.

According to one embodiment, in step f) above the at least two receiving pockets are filled by two separate filling lances with a liquid, solid and/or pasty content that is, e.g., designed for the production of dialysate.

In some embodiments, a multi-chamber bag described herein and its butt-welded connection is made of a polymer material and is designed as a single-use item or a disposable.

In some embodiments, a method described herein is carried out with a single machine.

At this point, it should be noted that the present disclosure is not limited to the explicitly disclosed combinations of features, but that the features disclosed herein may also be claimed in other combinations or in isolation.

Further advantages, effects, and features of the present disclosure are shown in the following description of embodiments with reference to the drawings, in which identical reference symbols denote identical or similar components.

### BRIEF DESCRIPTION OF THE FIGURES

In the drawings:

FIG. 1 shows a multi-chamber bag, and

FIG. 2 shows a continuous production of a multi-chamber bag from a continuous film tube.

### DETAILED DESCRIPTION

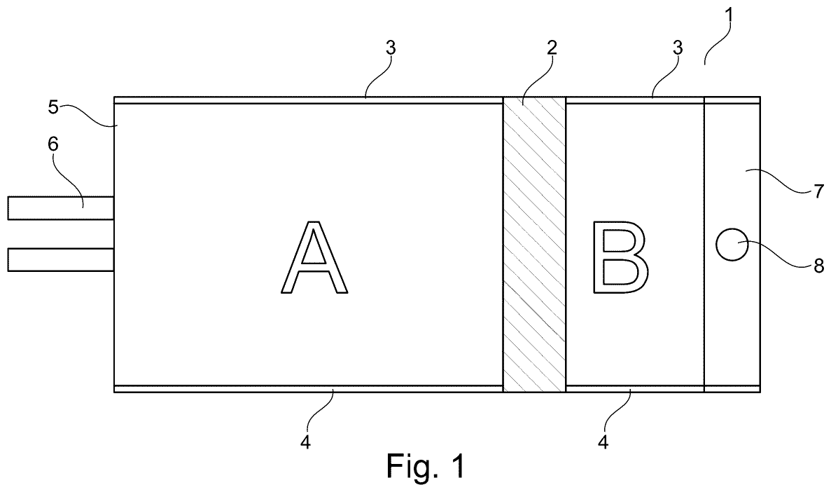

FIG. 1 shows a multi-chamber bag 1 having two chambers A and B, which are connected to each other by a peel seam 2 and which are each limited on a first and a second opposite side 3 and 4 by a respective sealing seam.

The multi-chamber bag 1 also comprises a third side 5 formed by a folded-over film, on which two connections in the form of tubular sections 6 are arranged by means of butt welding.

The bag 1 is produced from a flat film by folding the film over on the third side 5 and sealing the outer edges of the flat film together on a fourth side to form a sealing seam 7, which initially creates a film tube. At least one suspension eyelet 8 is located in the sealing seam 7, which is opposite the connections 6.

The transverse sealing seam shown in FIG. 1 at the lower end (second side 4) of the bag 1 is then created.

In this intermediate state, the chambers A and B are still open on their upper side in FIG. 1 and thus form receiving pockets that can be conveniently filled from above (in the view shown in FIG. 1).

After filling the receiving pockets, the transverse sealing seam shown in FIG. 1 at the upper end (first side 3) of the bag 1 is created, whereby the receiving pockets are closed and the chambers A and B are formed.

The chamber B does not have its own connection or port, so that the content of the chamber B can only reach the outside via the open peel seam 2, the chamber A and its connections.

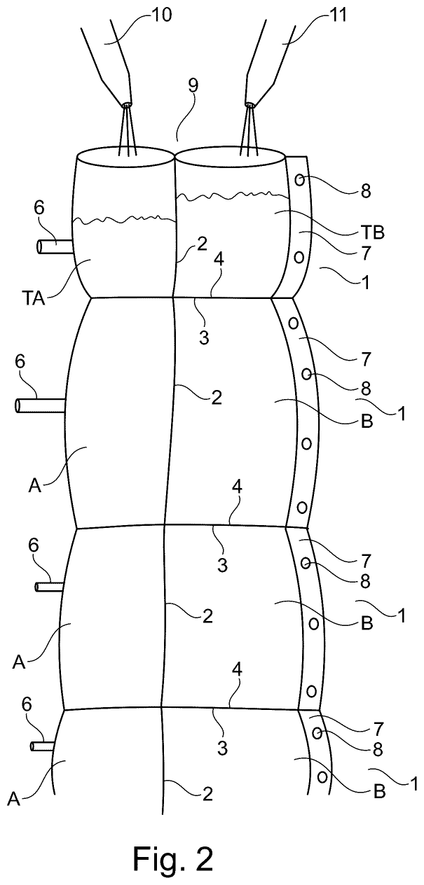

FIG. 2 shows a continuous production of a multi-chamber bag 1 according to the invention from a continuous film tube 9.

As shown in FIG. 2, a film tube 9 can extend in a vertical direction during filling. In the view shown in FIG. 2, the film tube 9 comprises four multi-chamber bags 1, which are not yet separated from each other in this view, but are each connected to each other by a common transverse sealing seam on the sides 3, 4.

The common transverse sealing seam on the sides 3, 4 separates the chambers A and B of adjacent multi-chamber bags 1. Each bag 1 is equipped with a butt-welded connection 6.

The top bag shown in FIG. 2 illustrates the filling of the chambers A and B. In this bag, the chambers A and B are not yet closed at their upper end shown in FIG. 2 and therefore form receiving pockets TA and TB for the filling material.

Each receiving pocket TA, TB is filled with a specific content by means of a separate filling device (e.g., a filling lance 10, 11), e.g., from above in the vertical direction. Once the receiving pockets TA and TB are sufficiently filled, they are closed by transverse sealing.

This type of filling eliminates the need to equip the chambers A and B of the multi-chamber bag with one filling connection or port each.

To separate the individual bags 1 of the continuous film tube 9 from each other, they are cut apart in the transverse direction along the common transverse sealing seam on the sides 3, 4.

1-11. (canceled)

12. A multi-chamber bag for producing a medical solution, the multi-chamber bag comprising:
at least two chambers that are connected to each other via a peel seam and that are delimited by a respective sealing seam at a first side and at a second side opposite the first side; and
a third side formed by a folded-over film, wherein at least one connection is formed on the third side using butt welding.

13. The multi-chamber bag of claim 12, wherein the medical solution comprises dialysate.

14. The multi-chamber bag of claim 12, comprising a sealing seam on a fourth side opposite the third side.

15. The multi-chamber bag of claim 14, wherein the fourth side comprises a suspension eyelet.

16. The multi-chamber bag of claim 12, wherein the multi-chamber bag does not comprise a filling port for filling at least one chamber of the at least two chambers of the multi-chamber bag.

17. The multi-chamber bag of claim 12, wherein the multi-chamber bag does not comprise a connection, port, or connector for filling at least one chamber of the at least two chambers of the multi-chamber bag.

18. The multi-chamber bag of claim 12, wherein a first chamber of the at least two chambers is at least partially limited by the third side comprising the connection, and wherein a second chamber of the at least two chambers is configured to be connected to the first chamber by opening the peel seam, wherein a content of the second chamber can only be transferred to an outside environment through the first chamber and the at least one connection on the third side.

19. The multi-chamber bag of claim 18, wherein the second chamber is limited exclusively by the peel seam, the sealing seams on the first and second sides, and the sealing seam on the fourth side.

20. The multi-chamber bag of claim 12, wherein a longitudinal axis of the peel seam extends at a right angle a right angle to a longitudinal axis of the multi-chamber bag.

21. The multi-chamber bag of claim 12, wherein the at least two chambers of the multi-chamber bag each have a content, wherein each content is designed to yield ready-to-use dialysate when the content of a respective chamber of the at least two chambers is combined with the content of another chamber of the at least two chambers of the multi-chamber bag.

22. The multi-chamber bag of claim 12, wherein the at least one connection is welded to the third side in such a way that the at least one connection sits on an outermost layer of the folded-over film and is not arranged between two films or integrated into the folded-over film or a layer structure of the folded-over film.

23. A method for producing a multi-chamber bag, wherein the method comprises:
providing a flat film with two outer edges;
butt welding at least one connection to the flat film;
producing a film tube out of the flat film by sealing the outer edges of the flat film together;
producing a peel seam that extends between two outer edges of the film tube along a longitudinal direction of the film tube, wherein at least two receiving areas are separated from each other by the peel seam;
producing a sealing seam that extends perpendicular or substantially perpendicular to the peel seam, wherein a receiving pocket is produced in each of the two receiving areas;
filling the two receiving pockets along the longitudinal direction of the film tube; and
closing the two receiving pockets by producing an additional sealing seam that extends perpendicular or substantially perpendicular to the peel seam, whereby the receiving pockets are closed and at least two completely closed chambers are formed.

24. The method of claim 23, wherein filling the two receiving pockets comprises filling the two receiving pockets with two different contents.

25. The method of claim 23, comprising producing multiple multi-chamber bags, wherein the multi-chamber bags are produced continuously along a continuous film tube, wherein the multiple multi-chamber bag of the multi-chamber bags are connected to each other by a sealing seam, and the multi-chamber bags are separated from each other by separation along the sealing seam extending perpendicular to the peel seam.

26. The method of claim 23, wherein filling the two receiving pockets comprises filling the two receiving pockets by two separate filling lances with at least one of a liquid, solid, or pasty content, wherein the content is configured for the production of dialysate.

27. The method of claim 23, comprising forming a suspension eyelet on the sealing seam or on the additional sealing seam.

28. The method of claim 23, wherein a first receiving pocket of the two receiving pockets is configured to be fluidly connected to a second receiving pocket of the two receiving pockets by opening the peel seam, wherein a content of the first receiving pocket can only be transferred to an outside environment through the second receiving pocket and the at least one connection.

29. The method of claim 23, wherein producing the peel seam comprises producing the peel seam in a way such that the at least one connection sits on an outermost layer of the film tube and is not arranged between two films or integrated into a folded-over film or a layer structure of the folded-over film.

30. The method of claim 23, wherein a welding temperature for fixing the at least one connection to the flat film is in a range of 150° C. to 300° C.

31. The method of claim 23, wherein a welding time for fixing the at least one connection to the flat film is in a range of 0.7 seconds to 2 seconds.
